(19) United States
(12) Patent Application Publication
(10) Pub. No.: US 2004/0162527 A1
(43) Pub. Date: Aug. 19, 2004

(54) Sterile sheath for an injection syringe

(76) Inventors: Gerhard Moersdorf, Munchweiler/Alsenz (DE); Herbert Maslanka, Tuttlingen (DE)

Correspondence Address: Finnegan, Henderson, Farabow, Garrett & Dunner, L.L.P., 1300 I Street, N.W., Washington, DE, 20005-3315, US

(21) Appl. No.: 10/758,261

(22) Filed: Jan. 16, 2004

(30) Foreign Application Priority Data
Jan. 17, 2003 (DE) 10301884.0

(51) Int. Cl. A61M 5/00

(52) U.S. Cl. 604/199

(57) ABSTRACT

The invention relates to a sterile sheath for an injection syringe to guarantee sterility for a product which has been taken up by the injection syringe. The sterile sheath includes a sealable casing made of plastic and an output connection piece, the interior of the sheath serving to receive the injection syringe. The injection syringe can be connected to the output connection piece, which is provided with a valve whose direction of flow is exclusively from the injection syringe to the exterior. Owing to the valve, no microorganisms are able to ascend from the exterior into the syringe.

The invention relates to a sterile sheath for an injection syringe. When such a sterile sheath is used, an injection syringe can be kept aseptic for several administrations.

On occasion, the medicine which is to be administered to the patient by means of an injection syringe is measured out so generously that the entire content of the injection syringe is not required. However, it is only possible to store the residual medicine, which remains in the injection syringe, for a subsequent use in the same or a different patient, if the injection syringe is stored until then in a sterile manner.

DE 299 20 664 U1 discloses a sterile sheath for an injection syringe. The sterile sheath includes a transparent and sealable plastic pocket which serves to receive the injection syringe. The plastic pocket is equipped with a disposable needle for administering the medicament. The end opposite the disposable needle can be sealed by means of an adhesion closure. The plastic pocket is suitable for receiving two injection syringes for administering a tissue adhesive. One injection syringe contains a fibrinogen solution and the other thrombin solution, and either a separate needle is provided for each of these two solutions or else a joint double needle is provided.

In the case of such a sterile sheath, with an injection syringe and a disposable needle, there is the danger of microbial contamination from bacteria ascending from the end of the needle and up into the injection syringe. This is not a problem when the equipment is used as directed, such that for the injection syringe is removed immediately after administration.

One object of the invention is to design a sterile sheath for an injection syringe, the sheath ensuring optimal sterility of the product which is taken up by the injection syringe.

For the purpose of achieving this object, the invention proposes a sterile sheath for an injection syringe having the following features:

the sterile sheath includes a sealable casing made of plastic,

the sterile sheath possesses an output connection piece,

the interior of the sterile sheath serves to receive the injection syringe, which can be connected to the output connection piece,

the output connection piece is provided with a valve whose direction of flow is from the injection syringe to the exterior.

A feature of the sterile sheath is that its output connection piece is additionally provided with the valve. The user is thereby guaranteed that the equipment will be as safe to use as possible. No problem arises even if the injection syringe is not removed from the output connection piece of the sterile sheath immediately following the administration since, because of the valve, no bacteria are able to ascend into the syringe from the exterior.

A medical device, in particular a needle, an adapter, a multiport valve or an infusion bottle, can be connected to the outer region of the output connection piece.

The casing is at least partially transparent in at least some areas. It can, of course, also be completely transparent, in order to be able to satisfactorily view and handle the injection syringe which the sterile sheath encloses.

The direction of flow through the valve, from the injection syringe to the exterior, can be effected in a variety of ways: preferably, the valve is designed as a non-return valve or as a duckbill valve. In principle, it is conceivable to provide a valve which is open in both directions, as is used in the case of infusion bottles. Such a valve, which is open in both directions, has a pressure body in the center, which body only opens when a particular pressure has been exceeded. It is possible to use this valve type because the pressure is only exceeded under the influence of the injection syringe and not as a result of the medical device, for example the needle, which can be connected to the output connection piece.

One embodiment of the valve provides for the output connection piece possessing at least one radial discharge aperture which can be sealed by means of an elastic ring element, in particular a tubular ring element, which encloses the output connection piece.

Expediently, the output connection piece receives both the injection syringe and the medical device, in particular the needle. When commercially available injection syringes are used, the output connection piece possesses a cone-shaped recess which serves to receive the syringe cone of the injection syringe. When a customary disposable needle is used, the output connection piece possesses a cone-shaped part for receiving a cone-shaped recess in the disposable needle. On the other hand, a swivel closure can be provided for connecting the output connection piece to the medical device, for example in the nature of a luer-lock closure or of a screw thread. The output connection piece thus possesses a threaded part for receiving a threaded part of the medical device.

One embodiment provides for the output connection piece to be designed as a hollow body. The dimensions of the hollow body are preferably such that the hollow body essentially serves to receive the cylindrical section of the injection syringe. The hollow body then essentially has a cylindrical shape. The injection syringe handle, which extends transversely to the syringe plunger, is arranged outside the hollow body so that it can be grasped conveniently.

In order to be able to administer the product, the sterile sheath is designed as a film, and is flexible in the region facing away from the output connection piece such that the injection-syringe can be operated in the customary manner.

One embodiment of the invention provides for the sterile sheath to be formed by the output connection piece and a pressure pocket. The rigid output connection piece is, in particular, designed as an injection molding part made of plastic. The pressure pocket should also be made of plastic and is designed, for example, as an injection molding part. However, it may be advantageous if the pressure pocket is formed by a shoulder piece, in particular a rigid shoulder piece, for connecting to the output connection piece, and a film-like plastic hood.

Depending on the numbers in which the sterile sheath according to the invention is produced, the pressure pocket can also be produced in a dipping method or by means of extrusion-blow molding, instead of as a plastic injection molding part.

In order to ensure a secure, detachable connection of the shoulder piece and the output connection piece, the shoulder piece is preferably provided with a snap-in lug, in particular a circumferential snap-in lug, which can be brought into engagement behind a corresponding snap-in lug of an annular plate belonging to the output connection piece.

Because of the configuration of the injection syringe, with handle ribs which extend perpendicularly to the direction in which the syringe plunger is displaced, it is considered to be advantageous if the output connection piece exhibits an oval ring aperture in the region of its annular plate which faces away from the valve, and the pressure pocket, in particular its shoulder piece, essentially encloses an oval.

In addition, a sealing element, which is designed, for example, as a sealing lip, is advantageously provided for the purpose of sealing in the region in which the output connection piece and the pressure pocket are connected. Other features of the invention are presented in the claims, the description of the figures and the figures themselves.

The accompanying drawings, which are incorporated in and constitute a part of this specification, illustrate one embodiment of the invention, and together with the description, serve to explain the principles of the invention.

### BRIEF DESCRIPTION OF THE DRAWINGS

FIG. 1 shows the sterile sheath according to the claimed invention, together with the injection syringe and disposable needle, in the assembled state, as seen from the side;

FIG. 2 shows the organization of the sterile sheath, together with the injection syringe and the disposable needle, in a view as shown in FIG. 1 but as an exploded representation;

FIG. 3 shows an enlarged representation of the output connection piece, as seen from the side;

FIG. 4 shows a detail Z of the output connection piece illustrated in FIG. 3;

FIG. 5 shows the pressure pocket forming a component of the sterile sheath, in a side view;

FIG. 6 shows the pressure pocket shown in FIG. 5 in the region in which it is connected to the output connection piece;

FIG. 7 shows the pressure pocket in a view VII in accordance with FIG. 5; and

FIG. 8 shows the output connection piece in a view VIII in accordance with FIG. 3.

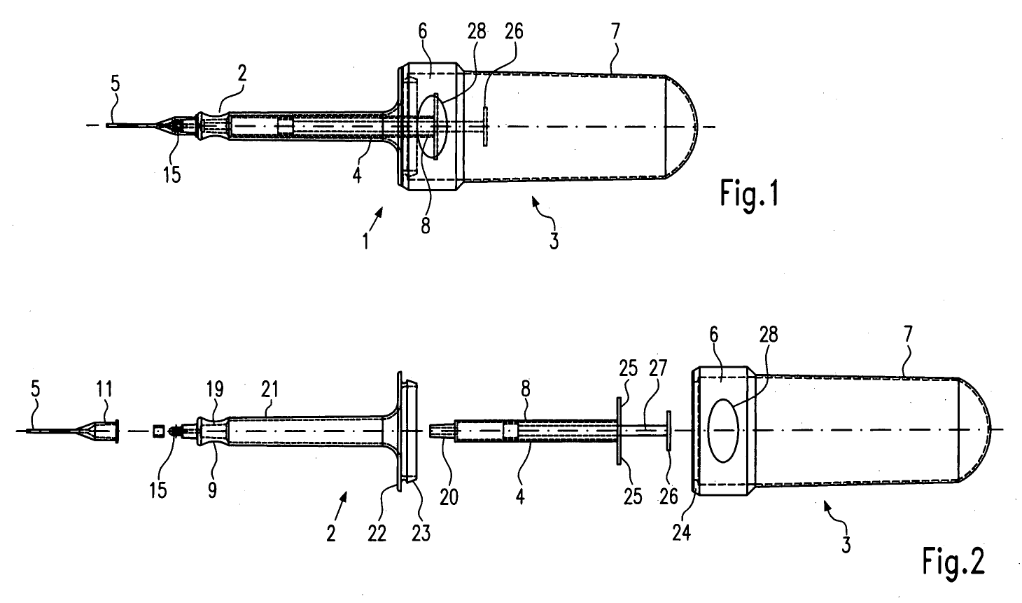

The sterile sheath 1 is formed by an output connection piece 2 and a pressure pocket 3 which can be connected to each other in a snap-in manner. Based on the function of the sterile sheath 1, an injection syringe 4 can be fitted into the inside of the output connection piece 2 and a disposable needle 5 can be fitted onto the outside of the output connection piece 2. The output connection piece 2 constitutes a rigid constructional element of plastic and is preferably produced by injection molding. The pressure pocket 3 is also formed as a plastic injection molding part. It is formed by a rigid shoulder piece 6 and a film-like plastic hood 7 which is connected to it. The shoulder piece 6 can be connected to the output connection piece 2 in a snap-in manner.

Because of the flexible configuration of the plastic hood 7, the injection syringe 4 can be actuated from the outside without having to be grasped directly.

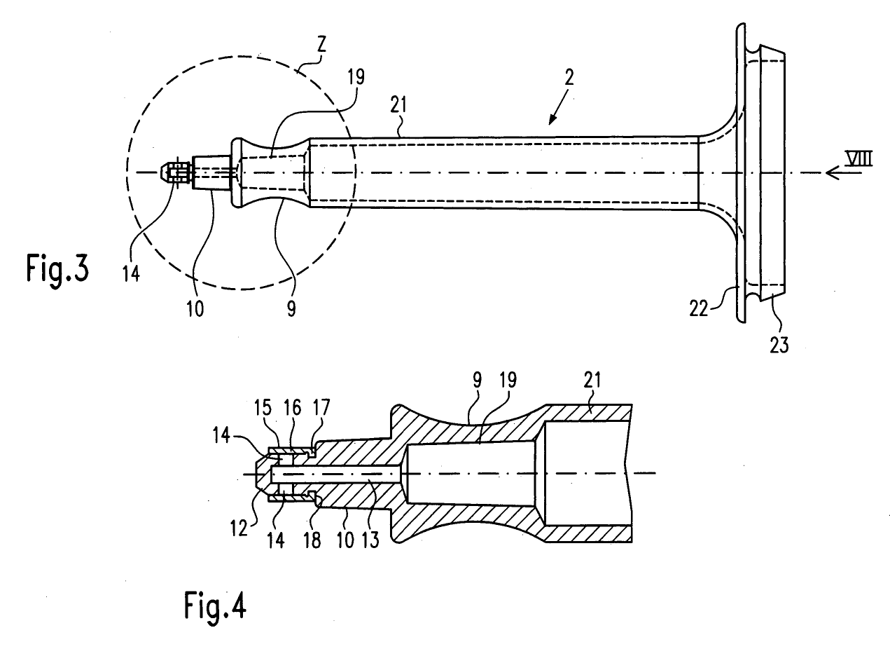

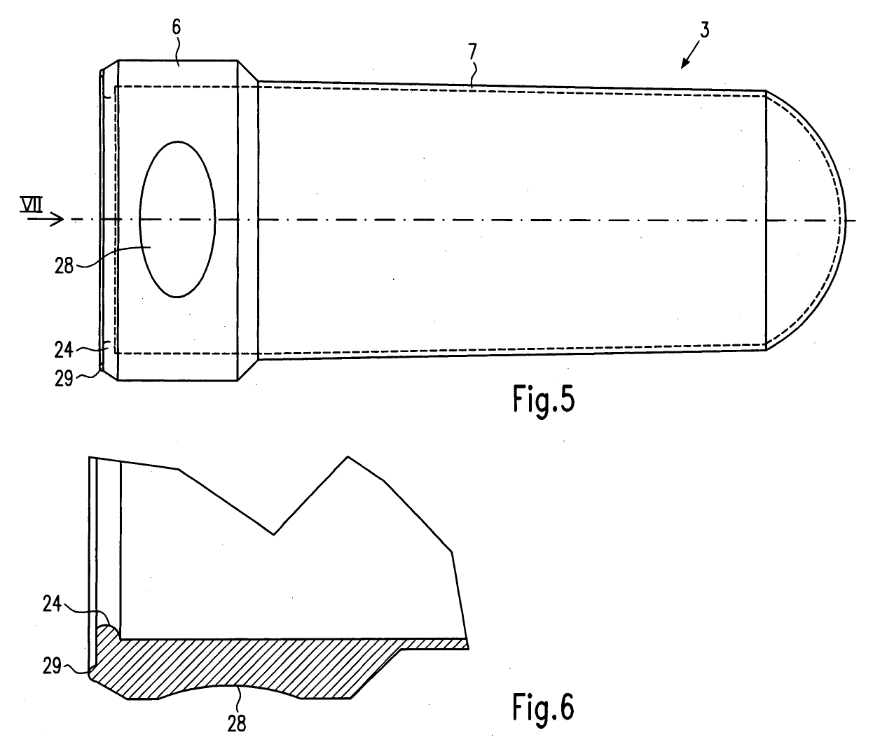

The output connection piece 2 is designed as a hollow body which essentially serves to receive the cylindrical section 8 of the injection syringe 4. As can be seen from the diagrams shown in FIGS. 3 and 4, in particular, the output connection piece 2 possesses, in its front region, a circumferential recessed grip 9 to which is connected, toward the front, a conically tapering section 10 for receiving a conically shaped recess 11 in the disposable needle 5. The section 10 and a section 12 of the output connection piece 2 which is arranged still further toward the front are provided with an axial drilled hole 13 which is sealed at the end and which opens out, in the region of the section 12, into a drilled hole 14 which traverses the section 12 region radially. The two drilled hole apertures of the drilled hole 14 are closed by means of a non-return valve 15. Non-return valve 15 possesses a flexible plastic ring 16, which may be made of silicone, for example, and which extensively encircles the mantle of the section 12 and occludes the openings of drilled hole 14 by exerting tension. Adjacent to the section 10, the plastic ring 16 possesses a circumferential nose section 17 which is directed inward and which engages in an undercut 18 of the section 12 and thereby functions as axial security for the plastic ring 16.

If a medicament, for example fibrinogen or thrombin, is administered through the drilled holes 13 and 14, the plastic ring 16 rises slightly from the section 12, thereby opening the non-return valve 15. Because of the function of the non-return valve 15, it is not possible for the medicament to pass in the opposite direction.

In the region of the recessed grip 9, the output connection piece 2 is provided internally with a cone-shaped recess 19 which serves to receive the syringe cone 20 of the injection syringe 4. The internal diameter of the hollow-cylindrical section 21 of the output connection piece 2 is dimensioned such that it is sufficiently larger than the outer diameter of the cylindrical section 8 of the injection syringe 4 such that the cylindrical section 8 can be introduced, with clearance, into the section 21 of the output connection piece 2 and can, by means of its syringe cone 20, be inserted into the recess 19 of the output connection piece 2.

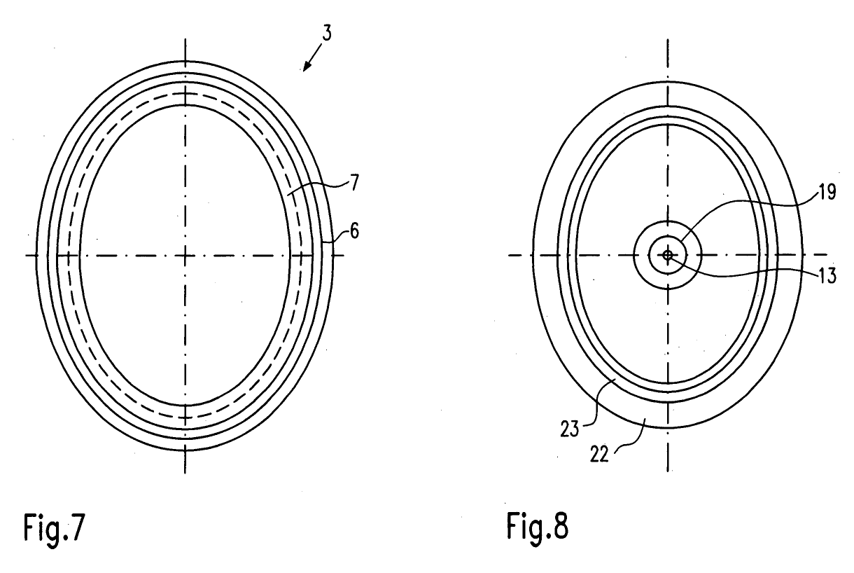

As compared with the section 21 of the output connection piece 2, the end of the output connection piece 2 facing away from the section 12 is designed such that it is markedly broader and configured in the form of an annular plate 22. As can be seen from the diagram in FIG. 8, the plate 22 has an oval shape and is provided with a snap-in lug 23 which is correspondingly located ovally. Snap-in lug 23 can be brought into functional linkage with a snap-in lug 24 of the shoulder piece 6 of the pressure pocket 3. In this respect, the shoulder piece 6 also has an oval shape, as can be seen from the diagram in FIG. 7. The flexible plastic hood 7 forms a structural unit together with the shoulder piece 6. The oval configuration of the previously described structural components takes into account the radial extension of the injection syringe 4 in the region of the diametrically opposed handle brackets 25 which are connected to the cylindrical section. The reference number 26 denotes the handle bracket which is connected to the plunger bar 27 of the injection syringe 4. In the region of the two side walls, the shoulder piece 6 is provided with recessed grips 28 in order to be able to grasp the sterile sheath 1 as a whole more conveniently.

In the region of the nose section 17 and the undercut 18 of the output connection piece 2, a seal is formed between the plastic ring 16 of the non-return valve 15 and the sections 10 and 12 of the output connection piece 2. Another seal is formed between the shoulder piece 6 and the annular plate 22 of the output connection piece 2 as a result of a circumferential sealing lip 29 belonging to the shoulder piece 6.

The described sterile sheath 1 consequently includes two injection-molded parts which are to be connected imperviously to each other. The non-return valve 15, which only opens at a pressure which has been previously set (during construction) and prevents any backflow into the injection syringe 4 following administration, is attached at the discharge end. The disposable needle 5 can be coupled on either by way of a normal cone or by way of a luer-lock connection. The user is free to choose the needle. The medicament can only be administered when the pressure exerted on the plunger bar 27 of the injection syringe 4 is appropriate. The intensity of the pressure required for opening the non-return valve 15 can be adjusted in advance by altering, for example, the diameter of the discharge drilled hole 14, the strength/elasticity of the sealing plastic ring 16, the nature of the valve, etc. The valve prevents any ascent of liquid and consequently of microorganisms.

| | Reference number list |
|---|---|
| Sterile sheath | 1 |
| Output connection piece | 2 |
| Pressure pocket | 3 |
| Injection syringe | 4 |
| Disposable needle | 5 |
| Shoulder piece | 6 |
| Plastic hood | 7 |
| Cylindrical section | 8 |
| Recessed grip | 9 |
| Section | 10 |
| Recess | 11 |
| Section | 12 |
| Drilled hole | 13 |
| Drilled hole | 14 |
| Non-return valve | 15 |
| Plastic ring | 16 |
| Nose section | 17 |
| Undercut | 18 |
| Recess | 19 |
| Syringe cone | 20 |
| Section | 21 |
| Plate | 22 |
| Snap-in lug | 23 |
| Snap-in lug | 24 |
| Handle bracket | 25 |
| Handle bracket | 26 |
| Plunger bar | 27 |
| Recessed grip | 28 |
| Sealing lip | 29 |

What is claimed is:

1. A sterile sheath for an injection syringe comprising:
a sealable casing made of plastic, the casing including
an output connection piece, wherein the output connection piece includes a valve whose direction of flow is exclusively from an injection syringe to the exterior, wherein an interior of the sterile sheath is configured to receive the injection syringe, and wherein the output connection piece is configured to be connected to the syringe.

2. The sterile sheath of claim 1, wherein an outer region of the output connection piece is configured to receive a medical device, and wherein the medical device is one of a needle, an adapter, a multiport valve, and an infusion bottle.

3. The sterile sheath of claim 1, wherein the sealable casing is at least partially transparent.

4. The sterile sheath of claim 1, wherein the valve is one of a non-return valve and a duckbill valve.

5. The sterile sheath of claim 1, wherein the output connection piece includes at least one radial discharge aperture sealable by means of an elastic ring element.

6. The sterile sheath of claim 5, wherein the elastic ring element is a tubular ring element, which encloses a portion of the output connection piece.

7. The sterile sheath of claim 1, wherein the output connection piece includes a cone-shaped recess configured to receive a syringe cone of the injection syringe.

8. The sterile sheath of claim 1, wherein the output connection piece includes a hollow body configured to receive a cylindrical section of the injection syringe.

9. The sterile sheath of claim 8, wherein the hollow body has a cylindrical shape.

10. The sterile sheath of claim 1, wherein the output connection piece includes, on an end opposite from the valve, an annular plate.

11. The sterile sheath of claim 10, wherein the annular plate has an oval aperture.

12. The sterile sheath of claim 1, wherein the output connection piece is formed of a rigid plastic by injection molding.

13. The sterile sheath of claim 1, the sealable casing further comprising a pressure pocket configured to connect to the output connection piece.

14. The sterile sheath of claim 13, wherein the pressure pocket is formed of plastic by injection molding.

15. The sterile sheath of claim 13, wherein the pressure pocket is formed by a dipping method.

16. The sterile sheath of claim 13, wherein the pressure pocket is formed by means of extrusion-blow molding.

17. The sterile sheath of claim 13, wherein the pressure pocket includes a shoulder piece configured to connect to the output connection piece, and further includes a film-like plastic hood.

18. The sterile sheath of claim 17, wherein the shoulder piece includes a snap-in lug configured to engage a snap-in lug on an annular plate of the output connection piece.

19. The sterile sheath of claim 13, further comprising a sealing element to seal between the output connection piece and the pressure pocket.

20. The sterile sheath of claim 2, wherein the output connection piece includes a section configured as a cone to receive a cone-shaped recess of the medical device.

21. The sterile sheath of claim 2, wherein the output connection piece and the medical device are connected by means of a swivel closure.

22. The sterile sheath of claim 21, wherein the swivel closure is one of a luer-lock closure and a screw thread.

23. The sterile sheath of claim 1, the sealable casing further including a pressure pocket configured to connect to the output connection piece, wherein the output connection piece further includes at least one radial discharge aperture sealable by means of an elastic ring element; a cone-shaped recess configured to receive a syringe cone of the injection syringe; and an annular plate, on an end of the output connection piece opposite the valve, configured to connect to the pressure pocket.

24. A method of keeping an injection syringe aseptic, comprising:
drawing fluid into an injection syringe;
placing the injection syringe within an interior of a sterile sheath, wherein the sterile sheath comprises:
a sealable casing made of plastic, the casing including
an output connection piece wherein the output connection piece includes a valve whose direction of flow is exclusively from the injection syringe to the exterior, wherein an interior of the sterile sheath is configured to receive the injection syringe, and wherein the output connection piece is configured to be connected to the syringe;
connecting one end of the syringe to the output connection piece;
connecting the pressure pocket to the output connection piece; and
connecting a medical device to the outer region of the output connection piece.

25. The method of claim 24, further including operating the syringe enclosed in the sterile sheath to administer the fluid to a patient.

26. The method of claim 25, further including maintaining the syringe within the sterile sheath so long as fluid, intended to be used in subsequent administrations, remains in the syringe.

27. The method of claim 26, further including changing the medical device connected to the output connection piece prior to a subsequent administration.

28. The method of claim 27, wherein changing the medical device includes exchanging disposable needles.

29. The method of claim 24, wherein connecting the medical device includes connecting one of a needle, an adapter, a multiport valve, and an infusion bottle to the output connection piece.

30. The method of claim 24, wherein drawing fluid into the syringe includes drawing a medication into the syringe.

31. A sterile sheath for an injection syringe, comprising:
a pressure pocket having a shoulder portion and a hood portion;
an output connection piece having first and second ends and including a hollow body, a one way valve disposed on one of said ends, and a plate portion on the other of said ends, wherein said plate portion is configured to connect to said shoulder portion to create a sealed casing, and wherein said hollow body is configured to receive an injection syringe therein.

32. The sterile sheath of claim 31, wherein the end of the output connection piece with the valve is configured to be connected to a medical device.

33. The sterile sheath of claim 32, wherein the medical device is one of a needle, an adapter, a multiport valve, and an infusion bottle.

34. The sterile sheath of claim 31, wherein at least a portion of at least one of said pressure pocket and said output connection piece is transparent.

35. The sterile sheath of claim 31, wherein the valve is one of a non-return valve and a duckbill valve.
